(19) United States
(12) Patent Application Publication
(10) Pub. No.: US 2004/0186282 A1
(43) Pub. Date: Sep. 23, 2004

(54) Synthesis and method of purification of cyclic nucleotide derivatives

(76) Inventors: Edward M. Chait, Greensboro, NC (US); Seung-Yong Choi, Greensboro, NC (US)

Correspondence Address: Charles W. Calkins, Esq., Kilpatrick Stockton LLP, 1001 West Fourth Street, Winston-Salem, NC, 27101-2400, US

(21) Appl. No.: 10/393,190

(22) Filed: Mar. 20, 2003

(51) Int. Cl. C07H 19/04

(52) U.S. Cl. 536/26.1

(57) ABSTRACT

The present invention relates to methods for the synthesis and purification of cyclic nucleotide derivatives. The present invention provides a method for separating a cyclic nucleotide derivative from a mixture resulting from a chemical reaction to produce a cyclic nucleotide derivative from a cyclic nucleotide. In one embodiment, this mixture may comprise a cyclic nucleotide derivative, a pyridine solvent, and at least one of an alkyl carboxylic acid, an alkyl acid halide, or an alkyl carboxylic acid anhydride. In another embodiment, this mixture may comprise a cyclic nucleotide derivative and at least one of an alkyl carboxylic acid, an alkyl acid halide, or an alkyl carboxylic acid anhydride.

### FIELD OF INVENTION

The present invention relates to methods for the synthesis and purification of cyclic nucleotide derivatives.

### BACKGROUND

Cyclic nucleotides are a group of compounds containing a heterocyclic base, a ribofuranose ring, and a phophodiester moiety. The biochemical significance of cyclic nucleotides lies in their effect upon metabolic regulation. For example, adenosine 3′,5′-cyclic monophosphate (cAMP) is the intracellular mediator of the action of a large number of extracellular mammalian hormones.

The activity of cyclic nucleotides and cyclic nucleotide derivatives makes them a potential pharmacological target and various applications in the fields of medicine are being developed. For example, researchers have discovered that cyclic nucleotide derivatives such as N⁶,2′-O-dibutyryl-adenosine-3′,5′-cyclic monophosphate sodium salt (db-cAMP-Na) can be used in aqueous solutions for organ preservation or maintenance. (See, U.S. Pat. Nos. 5,552,267 and 5,370,989). This application alone requires large amounts of cyclic nucleotides as each year over 15,000 organ transplants are performed in this country, and over 80,000 people may be on an organ transplant waiting list at any one time. Since adequate preservation of organs intended for transplantation is critical to the proper functioning of an organ following implantation, the need for large quantities of cyclic nucleotide derivatives such as db-cAMP-Na is anticipated.

### SUMMARY OF INVENTION

The present invention provides methods for synthesizing cyclic nucleotide derivatives such as db-cAMP-Na. The synthetic method can be used to produce cyclic nucleotide derivatives in large quantity and in high yield. The present invention also provides methods for separating a cyclic nucleotide derivative from a mixture resulting from a chemical reaction to produce a cyclic nucleotide derivative from a cyclic nucleotide.

In one embodiment of the present invention, a method for separating a cyclic nucleotide derivative from a mixture comprising a cyclic nucleotide derivative and at least one of an alkyl carboxylic acid, an alkyl acid halide, or an alkyl carboxylic acid anhydride, comprises: a) adding a dialkyl ether to the mixture comprising a cyclic nucleotide derivative and at least one of an alkyl carboxylic acid, an alkyl acid halide, or an alkyl carboxylic acid anhydride to produce an organic solution; b) adding a water solution to the organic solution to produce a two phase system; c) extracting the cyclic nucleotide derivative from the organic solution to produce an aqueous solution of the cyclic nucleotide derivative; and d) removing the aqueous solution of the cyclic nucleotide derivative from the organic solution.

In another embodiment of the present invention, a method for synthesizing a cyclic nucleotide derivative comprising: a) adding an alkyl acid anhydride or an alkyl acid halide to a solution comprising an ammonium salt of a cyclic nucleotide and a pyridine solvent to produce a reaction mixture comprising a cyclic nucleotide derivative; b) concentrating the reaction mixture by evaporating the pyridine solvent; c) adding a dialkyl ether to the reaction mixture to produce an organic solution; d) adding a water solution to the organic solution to produce a two phase system; e) extracting the cyclic nucleotide derivative from the organic solution to produce an aqueous solution of the cyclic nucleotide derivative; and f) removing the aqueous solution of the cyclic nucleotide derivative from the organic solution.

### DETAILED DESCRIPTION OF THE INVENTION

Cyclic nucleotide derivatives such as db-cAMP-Na can be extremely expensive. They are also unstable and susceptible to hydrolysis. With the development of medical applications that require large quantities of cyclic nucleotide derivatives, such as db-cAMP-Na, the need exists for a method to synthesize and purify cyclic nucleotide derivatives on a large scale.

Synthetic methods for the synthesis of cyclic nucleotide derivatives typically involve treating a suitable salt of a cyclic nucleotide in anhydrous pyridine with an excess of an alkyl acid anhydride or an alkyl acid halide. Depending on the reaction conditions, one or more groups on the cyclic nucleotide are acylated. After the reaction has reached a desired point with either one or two positions acylated, the excess alkyl acid anhydride or alkyl acid halide is hydrolyzed. The removal of the resulting alkyl acid from reaction mixtures containing a cyclic nucleotide derivative has traditionally being accomplished by evaporation, distillation, and/or complexation with calcium hydroxide.

The hydrolysis of the excess anhydride or acid halide followed by the removal of the resulting alkyl acid using evaporation or distillation has several disadvantages. For example, while waiting for water to hydrolyze the excess anhydride or acid halide, the cyclic nucleotide derivative is also susceptible to hydrolysis. Further, as the scale of the reaction increases, the period needed to hydrolyze the excess anhydride or acid chloride also increases, and thus, the yield of the cyclic nucleotide derivative may decrease. The hydrolysis of the excess anhydride or acid halide followed by the removal of the resulting alkyl acid using complexation also has several disadvantages. For example, when calcium hydroxide is added to a reaction mixture to complex with the hydrolyzed alkyl acids, a highly exothermic reaction occurs. On a small scale (<1 g), the heat generated by the addition of a complexing agent such as calcium hydroxide can be controlled, and the cyclic nucleotide derivatives can be protected from hydrolysis. As the scale of the reaction increases, it becomes more and more difficult to control the heat generated by the addition of calcium hydroxide, and as a result, yields of the cyclic nucleotide derivative may decrease.

The present invention provides methods for synthesizing cyclic nucleotide derivatives. The present invention also provides methods for separating a cyclic nucleotide derivative from a mixture resulting from a chemical reaction to produce a cyclic nucleotide derivative from a cyclic nucleotide. These methods incorporate one or more steps that separate a cyclic nucleotide derivative from other organic compounds without hydrolysis of excess anhydride or acid halide followed by evaporation, distillation, and/or complexation techniques that reduce the yields and reproducibility on a large scale. Rather than hydrolyzing the excess acid anhydride or acid halide and then removing the acid through evaporation, distillation, and/or complexation, the methods of the present invention create a two phase system by diluting the reaction mixture with an organic solvent that is not miscible in water such as, but not limited to, a dialkyl ether, and adding a water solution to the reaction mixture. The order of the steps of diluting with an organic solvent and adding an aqueous solution can be reversed. Once the two phase system is set up, the cyclic nucleotide derivative is extracted into the water solution. This method can quickly separate the cyclic nucleotide derivative from the majority of the excess alkyl acid anhydride, alkyl acid halide or alkyl acid in the crude reaction mixture. The resulting aqueous solution comprising the cyclic nucleotide derivative can be further purified without unnecessary hydrolysis or decomposition.

In one embodiment, the present invention provides methods for separating a cyclic nucleotide derivative from a mixture resulting from a chemical reaction to produce a cyclic nucleotide derivative from a cyclic nucleotide. This mixture may comprise a cyclic nucleotide derivative, a pyridine solvent, and at least one of an alkyl carboxylic acid, an alkyl acid halide, or an alkyl carboxylic acid anhydride. In another embodiment, this mixture may comprise a cyclic nucleotide derivative and at least one of an alkyl carboxylic acid, an alkyl acid halide, or an alkyl carboxylic acid anhydride.

As used herein “cyclic nucleotide derivative” refers to salts of chemical compounds comprising a purine or pyrimidine base, a ribofuranose ring, and a phosphodiester. The purine and pyrimidine bases include adenine, guanine, cytosine, thymine, cytosine, uracil, and derivatives thereof.

The counter ion present in the cyclic nucleotide derivatives may comprise alkali cations such as sodium, lithium, and potassium. The counter ion may also comprise ammonium ions. The ammonium ions may optionally comprise 1 to 4 alkyl groups optionally substituted with one or more substitutents comprising alkyl or alkylhydroxy groups. Such ammonium ions are represented by the formula N(R¹)₄ wherein R¹ comprises —H, —C₁-C₆ alkyl, or —(CH₂—CH(R²)—O)ₙ—H, wherein R² comprises —H, —CH₃, —CH₂CH₃, or —CH₂CH₂OH, and n equals 1 to 4. Specific examples of ammonium ions used in this invention include triethylammonium ion [HNEt₃]⁺.

The cyclic nucleotide derivatives further comprise at least one lipophilic group or side chain. The at least one lipophilic group may be attached to the primary amine on adenine, guanine, and cytosine bases, or the 2′-position of the ribofuranose ring. A lipophilic group includes any group which can protect the cyclic nucleotide derivative from hydrolysis and which can enable the cyclic nucleotide derivative to pass through a cell membrane. Examples of lipophilic groups include, but are not limited to, alkyl, alkene, alkyne, acyl, and aryl groups. In embodiments, the lipophilic group on the cyclic nucleotide derivative comprises acyl groups with 2 to 10 carbon atoms such as acetyl, propionyl, butyryl, isobutyryl, valeryl, isovaleryl, pivalyl, and caprionyl groups.

In various embodiments of the present invention, the cyclic nucleotide derivative may comprise a cyclic adenosine monophosphate ammonium salt, an N⁶,2′-O-diacyl-adenosine-3′,5′-cyclic monophosphate trialkylammonium salt, an N⁶,2′-O-dibutyryl-adenosine-3′,5′-cyclic monophosphate trialkylammonium salt, or an N⁶,2′-O-dibutyryl-adenosine-3′,5′-cyclic monophosphate triethylammonium salt.

As previously discussed, synthetic methods for the synthesis of cyclic nucleotide derivatives typically involve treating a suitable ammonium salt of a cyclic nucleotide in an anhydrous pyridine solvent with an excess of an alkyl acid anhydride or an alkyl acid halide. The alkyl acid anhydrides or an alkyl acid halides used in this chemical reaction include the respective derivatives of any C₁-C₉ linear or branched alkyl carboxylic acid. For example, the alkyl acid anhydrides or alkyl acid halides may be derivatives of acetic, propionic, butyric, isobutyric, valeric, isovaleric, pivalic, and caprionic acids. In preferred embodiments, the alkyl acid anhydride or alkyl acid halide is a derivative of butyric acid. The pyridine solvent used in this chemical reaction includes alkaloids comprising a pyridine structure. Examples of pyridine solvents include but are not limited to pyridine, 3- or 4-methylpyridine (3- or 4-picoline), and quinoline.

Once the chemical reaction has reached a desired end point, depending on whether mono- or di-acylation is desired, the cyclic nucleotide derivative can then be separated from the other compounds in the mixture using methods of the present invention. Typically, the pyridine solvent is removed by evaporation or distillation under reduced pressure. Once the pyridine is removed, the cyclic nucleotide derivative is separated from a mixture comprising the cyclic nucleotide derivative and at least one of an alkyl carboxylic acid, an alkyl acid halide, or an alkyl carboxylic acid anhydride through the following steps of: a) adding a dialkyl ether to the mixture comprising a cyclic nucleotide derivative and at least one of an alkyl carboxylic acid, an alkyl acid halide, or an alkyl carboxylic acid anhydride to produce an organic solution, b) adding a water solution to the organic solution to produce a two phase system, c) extracting the cyclic nucleotide derivative from the organic solution to produce an aqueous solution of the cyclic nucleotide derivative, and d) removing the aqueous solution of the cyclic nucleotide derivative from the organic solution.

Any dialkyl ether may be added to the mixture so long as it forms a two phase system with a water solution. Examples of potentially useful dialkyl ethers include diethyl ether and methyl tert-butyl ether.

In an embodiment, the ratio of a water solution to dialkyl ether added to the mixture ranges from about 0.5 to 1.0 parts water solution per part of dialkyl ether. In another embodiment, the extraction step is conducted at a temperature from 0 to 35° C., preferably from 15 to 25° C. Further, the water solution may have a pH other than 7 and may also include salts such as, but not limited to, sodium chloride and calcium chloride. The pH of the water solution may be adjusted with compounds such as, but not limited to, sodium carbonate, sodium bicarbonate, potassium carbonate, potassium bicarbonate, sodium hydroxide, potassium hydroxide, acetic acid, hydrochloric acid, and sulfuric acid. The water solution may be deionized pyrogen free water (i.e. water for injection).

To improve the extraction of the cyclic nucleotide derivative into the aqueous layer, the two phase system may be agitated before the phases are separated. The time taken to agitate is limited only by the need to reduce the potential for the cyclic nucleotide derivatives to hydrolyze in the aqueous phase. In one non-limiting embodiment, the two phase system is agitated from 1 to 10 minutes before the two phases are separated.

Other extraction techniques may be used to extract the cyclic nucleotide derivatives into the aqueous layer and separate it from the at least one of an alkyl carboxylic acid, an alkyl acid halide, or an alkyl carboxylic acid anhydride. For example, extraction techniques include, but are not limited to, single contact extraction (ie., batch), simple multistage contact extraction, countercurrent multistage extraction, true continuous countercurrent extraction, or continuous countercurrent extraction. Any of the extraction techniques disclosed in Perry et al., Chemical Engineers' Handbook, 5^{th} Edition (McGraw-Hill, 1973), and Lo et al., Handbook of Solvent Extraction, Reprint Edition (Krieger, 1991), can be used in the present invention.

The time taken to perform steps (a)-(d) is limited by the need to reduce the potential for the cyclic nucleotide derivative to hydrolyze in the aqueous phase. In one non-limiting embodiment, the time take to perform steps (a)-(d) ranges from 1 to 60 minutes. The time take to perform steps (a)-(d) may also range from 5 to 30 minutes.

The method of the present invention may include additional steps. For example, once the aqueous solution has been separated from the organic solution, the aqueous solution may be back extracted with dialkyl ether followed by the separation of the aqueous solution from the dialkyl ether. This back extraction step may be repeated several times to improve the separation of the cyclic nucleotide derivatives from the at least one of an alkyl carboxylic acid, an alkyl acid halide, or an alkyl carboxylic acid anhydride.

In another example of additional steps that may be included in the method of the present invention, once the aqueous solution has been separated from the organic solution, the water may be removed from the aqueous solution by evaporation or distillation to produce a cyclic nucleotide derivative residue. A quantity of water sufficient to dissolve the cyclic nucleotide derivative residue is then added to produce a residue solution followed by adding an ion exchange resin to the residue solution. Once the ammonium counter ion of the cyclic nucleotide derivatives is exchanged for the cation in the ion exchange resin, the ion exchange resin is removed from the solution. An aqueous solution of alkali base can then be added to the residue solution to obtain a pH of between 7.0 and 8.0, and the water is then removed from the residue solution by evaporation or distillation.

The evaporation or distillation steps are typically performed at a temperature of from 0 to 50° C., and at a pressure of from 0.01 to 100 torr. Preferably, the evaporation or distillation is performed at a temperature of from 15 to 25° C., and at a pressure of from 0.01 to 100 torr.

In summary, numerous benefits have been described which result from employing the concepts of the invention. The following Example of a preferred embodiment of the invention has been presented for purposes of illustration and description. It is not intended to be exhaustive or to limit the invention to the precise form disclosed. Obvious modifications or variations are possible in light of the above teachings. It is intended that the scope of the invention be defined by the claims appended hereto.

### EXAMPLE

The following reagents were obtained from the indicated commercial sources: andenosine-3′,5′-cyclic phosphoric acid (cAMP) was obtained from Acros. The triethylamine, pyridine, and butyric anhydride were obtained from Aldrich Chemical Co. The cationic exchange resin HCR-W2 was obtained from Dowex.

To a 5 liter round bottom flask was added andenosine-3′,5′-cyclic phosphoric acid (cAMP) (75 g, 0.228 mol, 1.0 eq.). At ambient temperature, a minimum amount of 0.4 M aqueous triethyl amine needed to dissolve the cAMP (typically around 500 ml, 0.228 mol, 1.0 eq.) was added. The mixture was manually agitated until a homogeneous solution was obtained. The homogeneous solution was concentrated to dryness under reduced pressure using rotary evaporation with a water bath temperature at no more than 50° C.

Anhydrous pyridine (500 ml) was then added to the 5 liter flask, and the mixture was manually agitated for 2 minutes. The resulting slurry was concentrated to dryness under reduced pressure using rotary evaporation with a water bath temperature at no more than 50° C. Anhydrous pyridine (1.2 L) was again added to the 5 liter flask, and the mixture was slowly heated to no more than 50° C. at standard pressure in a water bath to dissolve most of the cAMP triethylammonium salt.

After cooling the solution in an ice bath to approximately 30° C., butyric anhydride (1.0 L, 6.2 mol, 27 eq.) was added to the slurry while stirring. After five to eight days of stirring at room temperature (25° C.), the formation of the db-cAMP-triethylammonium salt was confirmed using HPLC analysis.

Under reduced pressure using rotary evaporation with a water bath temperature at no more than 50° C., the pyridine was removed from the reaction mixture. The solution was transferred into a separation flask having a mechanical stirrer attached.

Anhydrous diethyl ether (1.5 L) was added to the separation flask, and the mixture was mechanically stirred for 5 minutes. Next, water (1 L) was added to the separation flask, and the mixture was agitated for 3 minutes. After allowing the aqueous layer and ether layer to separate, the two layers were separated into different flasks. The aqueous layer was then back extracted two more times using anhydrous diethyl ether (1.5 L) each time.

The aqueous layer was concentrated to dryness under reduced pressure using rotary evaporation with a water bath temperature at no more than 50° C., with the resulting residue comprising db-cAMP-triethylammonium salt.

The triethylammonium ion of the db-cAMP-triethylammonium salt was exchanged for sodium using the following procedure. To the dried residue of db-cAMP-triethyammonium salt was added water (500 ml), and the mixture was manually agitated to make an aqueous solution. A cationic exchange resin (75 g) (HCR-W2, 16-40 mesh, spherical beads) was added to the aqueous solution. After agitating the mixture for 15 minutes, the ion exchange resin was removed using vacuum filtration. The pH of the filtered solution was adjusted to between 7.0 and 8.0 using 0.5 M sodium hydroxide.

The resulting solution of db-cAMP-Na was concentrated to dryness under reduced pressure using rotary evaporation with a water bath temperature at no more than 30° C., and then recrystallized with water and 1,4-dioxane by following a procedure similar to the one described in U.S. Pat. No. 4,015,066.

The yield of recrystallized db-cAMP-Na was approximately 56% (75 g) based on the initial amount of cAMP used. The scale of this procedure was doubled using 150 g of the cAMP starting material with a similar yield (55%).

We claim:

1. A method for separating a cyclic nucleotide derivative from a mixture comprising a cyclic nucleotide derivative and at least one of an alkyl carboxylic acid, an alkyl acid halide, or an alkyl carboxylic acid anhydride, said method comprising:
a) adding a dialkyl ether to the mixture comprising a cyclic nucleotide derivative and at least one of an alkyl carboxylic acid, an alkyl acid halide, or an alkyl carboxylic acid anhydride to produce an organic solution,
b) adding a water solution to the organic solution to produce a two phase system,
c) extracting the cyclic nucleotide derivative from the organic solution to produce an aqueous solution of the cyclic nucleotide derivative, and
d) removing the aqueous solution of the cyclic nucleotide derivative from the organic solution.

2. The method of claim 1, wherein the dialkyl ether comprises diethyl ether.

3. The method of claim 1, wherein the at least one of an alkyl carboxylic acid, an alkyl acid halide, or an alkyl carboxylic acid anhydride comprises butyric acid or butyric anhydride.

4. The method of claim 1, wherein the cyclic nucleotide derivative comprises cyclic adenosine monophosphate.

5. The method of claim 1, wherein the cyclic nucleotide derivative comprises N⁶,2′-O-diacyl-adenosine-3′,5′-cyclic monophosphate trialkylammonium salt.

6. The method of claim 1, wherein the cyclic nucleotide derivative comprises N⁶,2′-O-dibutyryl-adenosine-3′,5′-cyclic monophosphate trialkylammonium salt.

7. The method of claim 1, wherein the cyclic nucleotide derivative comprises N⁶,2′-O-dibutyryl-adenosine-3′,5′-cyclic monophosphate triethylammonium salt.

8. The method of claim 1, wherein about 0.5 to 1.0 parts water solution is added per part of dialkyl ether in the organic solution.

9. The method of claim 1, wherein the extraction step is conducted at from 0 to 35° C.

10. The method of claim 1, wherein the extraction step is conducted at from 15 to 25° C.

11. The method of claim 1, wherein the two phase system is agitated from 1 to 10 minutes before extracting the cyclic nucleotide derivative from the organic solution.

12. The method of claim 1, wherein the extraction step is a continuous, counter current extraction.

13. The method of claim 1, wherein the steps (a)-(d) are conducted from 1 to 60 minutes.

14. The method of claim 1, wherein the steps (a)-(d) are conducted from 5 to 30 minutes.

15. The method of claim 1, further comprising after removing the aqueous solution of the cyclic nucleotide derivative from the organic solution, extracting the aqueous solution with dialkyl ether, and removing the aqueous solution from the dialkyl ether.

16. The method of claim 1, further comprising after removing the aqueous solution of the cyclic nucleotide derivative from the organic solution, removing the water from the aqueous solution by evaporation or distillation to produce a cyclic nucleotide derivative residue, adding a quantity of water sufficient to dissolve the cyclic nucleotide derivative residue to produce a residue solution, adding an ion exchange resin to the residue solution, removing the ion exchange resin from the solution, adding an aqueous solution of alkali base to the residue solution to obtain a pH of between 7.0 and 8.0, and removing the water from the residue solution by evaporation or distillation.

17. The method of claim 16, wherein evaporation or distillation is performed at a temperature of from 0 to 50° C., and at a pressure of from 0.01 to 100 torr.

18. The method of claim 16, wherein evaporation or distillation is performed at a temperature of from 15 to 25° C., and at a pressure of from 0.01 to 100 torr.

19. A method for separating a cyclic nucleotide derivative from a mixture comprising a cyclic nucleotide derivative, a pyridine solvent, and at least one of an alkyl carboxylic acid, an alkyl acid halide, or an alkyl carboxylic acid anhydride, said method comprising:
a) concentrating the mixture comprising a cyclic nucleotide derivative, a pyridine solvent, and at least one of an alkyl carboxylic acid, an alkyl acid halide, or an alkyl carboxylic acid anhydride by evaporating the pyridine solvent,
b) adding a dialkyl ether to the mixture comprising a cyclic nucleotide derivative and at least one of an alkyl carboxylic acid, an alkyl acid halide, or an alkyl carboxylic acid anhydride to produce an organic solution,
c) adding a water solution to the organic solution to produce a two phase system,
d) extracting the cyclic nucleotide derivative from the organic solution to produce an aqueous solution of the cyclic nucleotide derivative, and
e) removing the aqueous solution of the cyclic nucleotide derivative from the organic solution.

20. A method for synthesizing a cyclic nucleotide derivative comprising:
a) adding an alkyl acid anhydride or an alkyl acid halide to a solution comprising an ammonium salt of a cyclic nucleotide and a pyridine solvent to produce a reaction mixture comprising a cyclic nucleotide derivative,
b) concentrating the reaction mixture by evaporating the pyridine solvent,
c) adding a dialkyl ether to the reaction mixture to produce an organic solution,
d) adding a water solution to the organic solution to produce a two phase system,
e) extracting the cyclic nucleotide derivative from the organic solution to produce an aqueous solution of the cyclic nucleotide derivative, and
f) removing the aqueous solution of the cyclic nucleotide derivative from the organic solution.

21. The method of claim 20, wherein the alkyl acid anhydride or alkyl acid halide are derivatives from acetic, propionic, butyric, isobutyric, valeric, isovaleric, pivalic, or caprionic acids.

22. The method of claim 20, wherein the alkyl acid anhydride or alkyl acid halide is a derivative of butyric acid.

23. The method of claim 20, wherein the pyridine solvent comprises pyridine.

24. The method of claim 20, wherein the ammonium salt of a cyclic nucleotide comprises cyclic adenosine monophosphate triethylammonium salt.

25. The method of claim 20, wherein about 0.5 to 1.0 parts water solution is added per part of dialkyl ether in the organic solution.

26. The method of claim 20, wherein the extraction step is conducted at from 0 to 35° C.

27. The method of claim 20, wherein the extraction step is conducted at from 15 to 25° C.

28. The method of claim 20, wherein the two phase system is agitated from 1 to 10 minutes before extracting the cyclic nucleotide derivative from the organic solution.

29. The method of claim 20, wherein the extraction step is a continuous, counter current extraction.

30. The method of claim 20, wherein the steps (c)-(f) are conducted from 1 to 60 minutes.

31. The method of claim 20, wherein the steps (c)-(f) are conducted from 5 to 30 minutes.

32. The method of claim 20, further comprising after removing the aqueous solution of the cyclic nucleotide derivative from the organic solution, extracting the aqueous solution with dialkyl ether, and removing the aqueous solution from the dialkyl ether.

33. The method of claim 20, further comprising after removing the aqueous solution of the cyclic nucleotide derivative from the organic solution, removing the water from the aqueous solution by evaporation or distillation to produce a cyclic nucleotide derivative residue, adding a quantity of water sufficient to dissolve the cyclic nucleotide derivative residue to produce a residue solution, adding an ion exchange resin to the residue solution, removing the ion exchange resin from the solution, adding an aqueous solution of alkali base to the residue solution to obtain a pH of between 7.0 and 8.0, and removing the water from the residue solution by evaporation or distillation.

34. The method of claim 33, wherein evaporation or distillation is performed at a temperature of from 0 to 50° C., and at a pressure of from 0.01 to 100 torr.

35. The method of claim 33, wherein evaporation or distillation is performed at a temperature of from 15 to 25° C., and at a pressure of from 0.01 to 100 torr.

36. A method for separating a cyclic nucleotide derivative from a mixture comprising a cyclic nucleotide derivative and at least one of an alkyl carboxylic acid, an alkyl acid halide, or an alkyl carboxylic acid anhydride, said method comprising:
creating a two phase system by adding a water immiscible organic solvent and a water solution to the mixture comprising a cyclic nucleotide derivative and at least one of an alkyl carboxylic acid, an alkyl acid halide, or an alkyl carboxylic acid anhydride,
extracting the cyclic nucleotide derivatives into the aqueous solution.
